(19) United States
(12) Patent Application Publication
(10) Pub. No.: US 2025/0304908 A1
(43) Pub. Date: Oct. 2, 2025

(54) CULTURE MEDIUM AND CULTURE METHOD FOR CONSTRUCTING PROSTATE CANCER ORGANOIDS

(71) Applicant: PLA Air Force Medical University, Xi'an (CN)

(72) Inventors: Xiang ZHANG, Xi'an (CN); Jiayun LIU, Xi'an (CN); Xiaoke HAO, Xi'an (CN); Yiting GAO, Xi'an (CN); Xinyang BU, Xi'an (CN); Manxin DU, Xi'an (CN); Wanting HUA, Xi'an (CN); Shengjia SHI, Xi'an (CN); Lijuan WANG, Xi'an (CN)

(73) Assignee: PLA Air Force Medical University, Xi'an (CN)

(21) Appl. No.: 18/985,073

(22) Filed: Dec. 18, 2024

(30) Foreign Application Priority Data
Mar. 27, 2024 (CN) 202410360399.8

(51) Int. Cl. C12N 5/00; C12N 5/071

(52) U.S. Cl. CPC C12N 5/0018; C12N 5/0683; C12N 2500/02; C12N 2500/38; C12N 2501/115; C12N 2501/119; C12N 2501/155; C12N 2501/415; C12N 2501/727; C12N 2501/999; C12N 2513/00; C12N 2523/00

(57) ABSTRACT

A culture medium and a culture method for constructing prostate cancer organoids are provided. The culture medium includes a basic culture medium, and the following components with final concentrations: R-spondin1 15-200 ng/mL, Noggin 20-150 ng/mL, FGF2 5-15 ng/mL, FGF10 1-10 ng/mL, CHIR99021 0.5-1 μM, ALK inhibitor 0.25-1 μM, ROCK inhibitor 5-30 μM, nicotinamide 5-10 mM and sodium pyruvate 0.5-3 mM. The present culture medium and culture method can effectively improve the survival rate and formation efficiency of organoids through the compounding of each component.

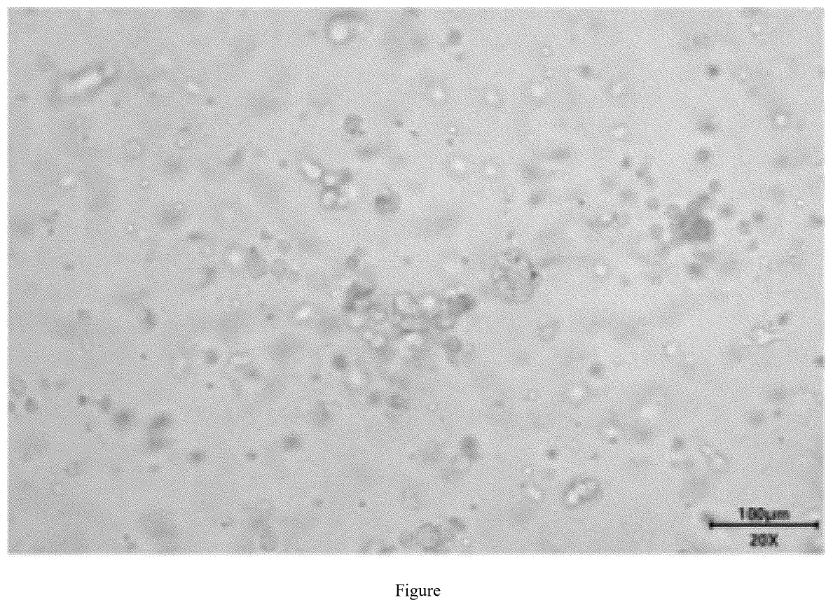

### CROSS REFERENCE TO THE RELATED APPLICATIONS

This application is based upon and claims priority to Chinese Patent Application No. 202410360399.8, filed on Mar. 27, 2024, the entire contents of which are incorporated herein by reference.

### TECHNICAL FIELD

The present disclosure belongs to the technical field of biotechnology, and in particular, relates to a culture medium and culture method for constructing prostate cancer organoids.

### BACKGROUND

Prostate cancer is one of the most common malignant tumors in the male reproductive system and ranks second among cancers that cause male death. In clinical diagnosis, malignant degrees of prostate cancer are closely related to the treatment effect of patients. Because early prostate cancer has no obvious symptoms, clinically diagnosed prostate cancer is mostly advanced, resulting in poorer treatment and prognosis of prostate cancer. At present, the conventional treatment for advanced prostate cancer is anti-androgen therapy, however, most patients will develop drug resistance after treatment, and eventually develop into castration-resistant prostate cancer (CRPC). Therefore, there is an urgent need for tools that can evaluate the efficacy of drugs before patients take medication.

Tumor organoid is a kind of cell culture that is derived from patient tumor tissue and can be cultured in three dimensions in vitro. The tumor organoids preserve the heterogencity, tissue characteristics and gene mutation information of tumors, and can simulate the occurrence and development of cancer in vitro. Therefore, constructing disease models by using the tumor organoids, and performing rapid and accurate screening of cancer drugs can effectively supplement culture models of existing animals and two-dimensional cells.

At present, when culturing prostate cancer organoids, it is mostly done by adding FBS serum and other methods. Such methods can have a greater impact on the nature of the organoids, and the growth and formation efficiency of the organoids is not good. Therefore, it is urgent to develop a new culture medium and culture method for prostate cancer organoids.

### SUMMARY

In view of the above shortcomings of the prior art, the present disclosure provides a culture medium and culture method for constructing prostate cancer organoids, which can effectively improve the formation efficiency and vital cell quantity of organoids.

In order to achieve the above objective, technical solutions adopted by the present disclosure to solve technical problems of the present disclosure are:
a culture medium for constructing prostate cancer organoids includes a basic culture medium, and the following components with final concentrations:
R-spondin1 15-200 ng/mL, Noggin 20-150 ng/ml, fibroblast growth factor (FGF) 2 5-15 ng/mL, FGF10 1-10 ng/mL, CHIR99021 0.5-1 μM, anaplastic lymphoma kinase (ALK) inhibitor 0.25-1 μM, Rho-associated protein kinase (ROCK) inhibitor 5-30 μM, nicotinamide 5-10 mM and sodium pyruvate 0.5-3 mM.

Further, the culture medium includes a basic culture medium, and the following components with final concentrations:
R-spondin1 200 ng/ml, Noggin 150 ng/ml, FGF2 5 ng/ml, FGF10 6 ng/ml, CHIR99021 0.5 μM, ALK inhibitor 0.5 μM, ROCK inhibitor 5 μM, nicotinamide 10 mM and sodium pyruvate 1.5 mM.

Further, the basic culture medium is Advanced Dulbecco's modified Eagle's medium/Ham's F12 (DMEM/F12).

Further, the ALK inhibitor is A83-01.

Further, the ROCK inhibitor is Y-27632.

A method for preparing the above culture medium includes adding each component to the basic culture medium and mixing evenly.

A culture method for prostate cancer organoids includes the following steps: co-culturing prostate cancer cells and prostate stromal cells by adopting the above culture medium.

Further, a specific process is as follows:
(1) acquiring precipitate of prostate cancer cells to be cultured;
(2) performing dripping adhesive and inoculation on the precipitate of cells, then adding prostate stromal cells, and incubating at 35-37° C. for 30-50 min; and
(3) adding the culture medium, and culturing at 35-37° C. and 5% CO₂ concentration for 5-7 d.

Further, in step (2), performing dripping adhesive and inoculation on the precipitate of prostate cancer cells of step (1) at a ratio of 1×10⁵-10⁶ cells/20 μL.

Further, in step (2), an inoculation amount of the prostate stromal cells is ⅓ of that of the prostate cancer cells.

The beneficial effects of the present disclosure are:
1. Rspondinl and Noggin used in the present disclosure can promote cell proliferation of organoids by promoting Wnt signal transduction and regulating BMP signal pathway, respectively, so as to improve the proliferation, invasion, migration and anti-apoptosis ability of prostate cancer organoids. At the same time, the combination of CHIR99021 and Noggin can enhance the self-renewal and proliferation ability of cells, and improve the formation efficiency of the organoids.
2. The FGF2 and FGF10 used in the formula of the present disclosure are helpful in promoting the proliferation of prostate cells, and A83-01 can promote the continuous proliferation of cells by inhibiting the TGF-β signaling pathway. After adding CHIR99021, CHIR99021 can prolong the culture time of the organoids together with A83-01. In addition, in the present disclosure, the compounding of Y-27632, A83-01 and CHIR99021 can effectively improve the cell survival rate and cell proliferation ability.
3. The addition of nicotinamide and sodium pyruvate also helps to improve the formation efficiency of the organoids. Moreover, A83-01, CHIR99021 and nicotinamide all have the effect of inhibiting cell differentiation, thus ensuring the stable construction of the organoids.
4. In the process of cultivating organoids, the present disclosure adds prostate stromal cells and co-cultures the prostate stromal cells with prostate cancer cells. By co-culturing with prostate stromal cells, the expression of AMCAR (α-methylacyl CoA racemase) can be effectively maintained, and the survival rate and formation efficiency of the organoids can be further improved.

### BRIEF DESCRIPTION OF THE DRAWINGS

FIGURE shows prostate cancer organoids obtained through cultivation in Embodiment 1.

### DETAILED DESCRIPTION OF THE EMBODIMENTS

In the following, the embodiments of the present disclosure will be described, to facilitate those skilled in the art to understand the present disclosure. However, it is to be clear that the present disclosure is not limited to the scope of the embodiments. For those skilled in the art, as long as various changes are within the spirit and scope of the invention defined and determined by the attached claims, these changes are obvious. All inventions utilizing the conception of the present disclosure are under protection.

### Embodiment 1

A culture medium for constructing prostate cancer organoids included a basic medium Advanced DMEM/F12, and the following components with final concentrations:

R-spondin1 200 ng/ml, Noggin 150 ng/mL, FGF2 5 ng/ml, FGF10 6 ng/ml, CHIR99021 0.5 μM, ALK inhibitor A83-01 0.5 μM, ROCK inhibitor Y-27632 5 μM, nicotinamide 10 mM and sodium pyruvate 1.5 mM.

Prostate cancer organoids were cultivated by using the above culture medium, and the specific process was as follows:
(1) collected prostate cancer tissues were washed with normal saline and then chopped, a digestive solution was added for digestion to obtain a product, and the product was diluted with HBSS buffer and centrifuged, to obtain precipitate of cells;
(2) the obtained precipitate of cells was resuspended in a matrigel, and dripping adhesive and inoculation were performed on the precipitate of cells at a ratio of 1×10⁵ cells/20 μL, then prostate stromal cells accounting for ⅓ of the amount of the precipitate of cells were added, and incubated at 37° C. for 30 min; and
(3) the above culture medium was added and cultured at 37° C., 5% CO₂ for 5 d.

### Embodiment 2

A culture medium for constructing prostate cancer organoids included a basic medium Advanced DMEM/F12, and the following components with final concentrations:

R-spondin1 200 ng/ml, Noggin 100 ng/ml, FGF2 5 ng/ml, FGF10 10 ng/ml, CHIR99021 0.5 μM, ALK inhibitor A83-01 0.5 μM, ROCK inhibitor Y-27632 15 μM, nicotinamide 6 mM and sodium pyruvate 0.5-3 mM.

The culture method of organoids in the present embodiment was the same as in Embodiment 1

### Embodiment 3

A culture medium for constructing prostate cancer organoids included a basic medium Advanced DMEM/F12, and the following components with final concentrations:

R-spondin1 180 ng/ml, Noggin 150 ng/mL, FGF2 10 ng/mL, FGF10 8 ng/ml, CHIR99021 0.5 μM, ALK inhibitor A83-01 0.25 μM, ROCK inhibitor Y-27632 6 μM, nicotinamide 8 mM and sodium pyruvate 0.5 mM.

The culture method of organoids in the present embodiment was the same as in Embodiment 1.

### Contrast Example 1

Compared with Embodiment 1, when cultivating prostate cancer organoids, the prostate stromal cells were not used for co-culture, and the other processes were the same as Embodiment 1.

### Contrast Example 2

Compared with Embodiment 1, the culture medium lacked nicotinamide, sodium pyruvate and CHIR99021, and the other processes were consistent with Embodiment 1.

### Contrast Example 3

Compared with Embodiment 1, SB202190 was adopted to replace CHIR99021 in the culture medium, L-glutamine was adopted to replace nicotinamide, and the other processes were consistent with Embodiment 1.

### Contrast Example 4

Organoids were cultured referring to culture kits of Organotial human prostate cancer organoids

### Experimental Example

### 1. Detection of the Number of Cells and Cell Viability

In the culture process of Embodiment 1 and Contrast Examples 1-4, the cell counting was performed on day 0, day 2, day 3 and day 5, respectively, the cell viability was detected on day 3 and day 5, and the results were shown in Table 1 and Table 2.

TABLE 1
Number of cells

| Number of cells | On day 0 | On day 2 | On day 3 | On day 5 |
|---|---|---|---|---|
| Embodiment 1 | 1 × 10⁵ | 3 × 10⁵ | 4 × 10⁶ | 4.8 × 10⁶ |
| Contrast Example 1 | 1 × 10⁵ | 1.8 × 10⁵ | 2.6 × 10⁵ | 3.3 × 10⁵ |
| Contrast Example 2 | 1 × 10⁵ | 1.4 × 10⁵ | 2.0 × 10⁵ | 2.5 × 10⁵ |
| Contrast Example 3 | 1 × 10⁵ | 2.1 × 10⁵ | 3.2 × 10⁵ | 3.5 × 10⁵ |
| Contrast Example 4 | 1 × 10⁵ | 2.0 × 10⁵ | 2.2 × 10⁵ | 3.0 × 10⁵ |

TABLE 2
Cell viability

| Cell viability | On day 3 (%) | On day 5 (%) |
|---|---|---|
| Embodiment 1 | 96.4 | 98.5 |
| Contrast Example 1 | 96.8 | 98.2 |
| Contrast Example 2 | 81.3 | 85.4 |
| Contrast Example 3 | 83.6 | 88.7 |
| Contrast Example 4 | 84.5 | 90.6 |

From the data recorded in Table 1 and Table 2, it could be seen that, in the present disclosure, the number of living cells and the cell viability of the organoids obtained by Embodiment 1 were significantly better than those of the Contrast Examples 1-4, indicating that when the co-culture method, or the key components such as nicotinamide, sodium pyruvate and CHIR99021 in the culture medium were replaced, the original synergistic system was destroyed, which significantly affected the efficacy of the culture medium.

### 2. Number and Size of Organoids

After 5 d of culture, the number and average size of the organoids were measured by randomly selecting five visual fields in the middle area under an ordinary optical microscope, and the results were shown in FIGURE and Table 3.

TABLE 3
Number and size of organoids

| | Embodiment | Contrast | Contrast | Contrast | Contrast |
|---|---|---|---|---|---|
| | 1 | Example 1 | Example 2 | Example 3 | Example 4 |
| Number of | 72 | 54 | 48 | 56 | 52 |
| organoids | | | | | |
| Size(μm) | 61 ± 5 | 42 ± 3 | 40 ± 3 | 44 ± 2 | 46 ± 4 |

It could be seen from the data in Table 3 that, in the present disclosure, the organoids cultivated in Embodiment 1 were significantly better than those in Contrast Examples 1˜4 on both quantity and size, indicating that only the culture medium system constructed by the present disclosure was most suitable for the growth of prostate cancer organoids.

### 3. Generations Test

The prostate cancer organoids cultivated by the technical solutions recorded in Embodiment 1 and Contrast Examples 1˜4 were performed generations tests respectively. Wherein, the prostate cancer organoids cultivated by the technical solutions of Embodiment 1 of the present disclosure might be passed down to 5-10 generations, and the organoids could still grow well at this time; while the prostate cancer organoids cultured by the technical scheme of Contrast Example 2 and Contrast Example 3 were apoptotic within 3 generations, and the culture failed; and the situations of Contrast Example 1 and Contrast Example 4 were slightly better, but still could not achieve the same technical effect as Embodiment 1 of the present disclosure.

Finally, it is to be explained that the above embodiments are merely used to explain the technical solutions of the present disclosure rather than the limitation. Although the present disclosure is described in detail with reference to examples, those skilled in the art should understand that the technical solutions of the present disclosure can be modified or replaced equivalently without deviating from the spirit and scope of the technical solutions of the present disclosure, which should be covered in the scope of claims of the present disclosure.

What is claimed is:

1. A culture medium for constructing prostate cancer organoids, comprising a basic culture medium and components with final concentrations as follows:
15-200 ng/ml of an R-spondin1, 20-150 ng/ml of Noggin, 5-15 ng/ml of a fibroblast growth factor (FGF) 2, 1-10 ng/mL of FGF10, 0.5-1 μM of CHIR99021, 0.25-1 μM of an anaplastic lymphoma kinase (ALK) inhibitor, 5-30 μM of a Rho-associated protein kinase (ROCK) inhibitor, 5-10 mM of nicotinamide, and 0.5-3 mM of sodium pyruvate.

2. The culture medium according to claim 1, wherein the culture medium comprises the basic culture medium and the components with the final concentrations as follows:
200 ng/ml of the R-spondin1, 150 ng/ml of the Noggin, 5 ng/ml of the FGF2, 6 ng/ml of the FGF10, 0.5 μM of the CHIR99021, 0.5 μM of the ALK inhibitor, 5 μM of the ROCK inhibitor, 10 mM of the nicotinamide, and 1.5 mM of the sodium pyruvate.

3. The culture medium according to claim 1, wherein the basic culture medium is Advanced Dulbecco's modified Eagle's medium/Ham's F12 (DMEM/F12).

4. The culture medium according to claim 1, wherein the ALK inhibitor is A83-01.

5. The culture medium according to claim 1, wherein the ROCK inhibitor is Y-27632.

6. A method for preparing the culture medium according to claim 1, comprising adding each of the components to the basic culture medium and mixing evenly.

7. A culture method for prostate cancer organoids, comprising the following steps: co-culturing prostate cancer cells and prostate stromal cells by adopting the culture medium according to claim 1.

8. The culture method according to claim 7, wherein a process is as follows:
(1) acquiring a precipitate of prostate cancer cells to be cultured;
(2) performing a dripping adhesive and an inoculation on the precipitate of the prostate cancer cells to be cultured, then adding the prostate stromal cells, and incubating at 35-37° C. for 30-50 min; and
(3) adding the culture medium, and culturing at 35-37° C. and 5% concentration of CO₂ for 5-7 d.

9. The culture method according to claim 8, wherein the step (2) comprises performing the dripping adhesive and the inoculation on the precipitate of the prostate cancer cells to be cultured of the step (1) at a ratio of 1×10⁵-10⁶ cells/20 μL.

10. The culture method according to claim 8, wherein in the step (2), an inoculation amount of the prostate stromal cells is ⅓ of an inoculation amount of the prostate cancer cells to be cultured.

11. The culture medium according to claim 2, wherein the basic culture medium is Advanced DMEM/F12.

12. The culture medium according to claim 2, wherein, the ALK inhibitor is A83-01.

13. The culture medium according to claim 2, wherein, the ROCK inhibitor is Y-27632.

14. The method according to claim 6, wherein the components added to the basic culture medium are as follows:
200 ng/mL of the R-spondin1, 150 ng/ml of the Noggin, 5 ng/ml of the FGF2, 6 ng/ml of the FGF10, 0.5 μM of the CHIR99021, 0.5 μM of the ALK inhibitor, 5 μM of the ROCK inhibitor, 10 mM of the nicotinamide, and 1.5 mM of the sodium pyruvate.

15. The method according to claim 6, wherein the basic culture medium is Advanced DMEM/F12.

16. The method according to claim 6, wherein in the culture medium, the ALK inhibitor is A83-01.

17. The method according to claim 6, wherein in the culture medium, the ROCK inhibitor is Y-27632.

18. The culture method according to claim 7, wherein the culture medium comprises the basic culture medium and the components with the final concentrations as follows:
200 ng/mL of the R-spondin1, 150 ng/mL of the Noggin, 5 ng/ml of the FGF2, 6 ng/ml of the FGF10, 0.5 μM of the CHIR99021, 0.5 μM of the ALK inhibitor, 5 μM of the ROCK inhibitor, 10 mM of the nicotinamide, and 1.5 mM of the sodium pyruvate.

19. The culture method according to claim 7, wherein in the culture medium, the basic culture medium is Advanced DMEM/F12.

20. The culture method according to claim 7, wherein in the culture medium, the ALK inhibitor is A83-01.
